(19) United States
(12) Patent Application Publication
(10) Pub. No.: US 2026/0224789 A1
(43) Pub. Date: Aug. 6, 2026

(54) ADJUSTABLE SUPPORT SYSTEM FOR A PERITONEAL DIALYSIS GRAVITY-BASED CYCLER

(71) Applicants: Fresenius Medical Care Holdings, Inc., Waltham, MA (US); FRESENIUS MEDICAL CARE DEUTSCHLAND GMBH, Bad Homburg (DE)

(72) Inventors: David Yuds, Hudson, NH (US); Stefan Perplies, Hunfeld (DE); Klaus Wolf, Arnstein (DE); Teresa Close, Waltham, MA (US); Lena Mandelka, Hochheim (DE)

(73) Assignees: Fresenius Medical Care Holdings, Inc., Waltham, MA (US); FRESENIUS MEDICAL CARE DEUTSCHLAND GMBH, Bad Homburg (DE)

(21) Appl. No.: 19/043,736

(22) Filed: Feb. 3, 2025

(51) Int. Cl. A61M 1/28; A61M 1/16

(52) U.S. Cl. CPC A61M 1/282; A61M 1/1621; A61M 2205/10

(57) ABSTRACT

The present disclosure describes systems and methods support system for adjusting a height of a gravity-based peritoneal dialysis device. In some embodiments, a support system for a gravity-based peritoneal dialysis device includes a base, a support platform, and one or more adjustable pillar assembly connected between the base and the support platform. The one or more adjustable pillar assembly can have a total length that is adjustable to correspondingly adjust a height of the support platform relative to the base.

### TECHNICAL FIELD

The present disclosure relates generally to dialysis systems and methods. More particularly, the present disclosure relates to systems and methods for adjusting a height of a peritoneal dialysis gravity-based cycler.

### BACKGROUND

Dialysis is a treatment used to support patients with insufficient renal function. The two principal dialysis methods are hemodialysis and peritoneal dialysis.

During hemodialysis (HD), the patient's blood is passed through a dialyzer of a dialysis machine while also passing a dialysis solution or dialysate through the dialyzer. A permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water, in the blood.

During peritoneal dialysis (PD), a patient's peritoneal cavity is periodically infused through a peritoneal dialysis catheter with a sterile aqueous solution, referred to as PD solution or dialysate. The membranous lining (peritoneum) of a patient's abdomen acts as a natural permeable membrane that allows diffusion and osmosis exchanges to take place between the dialysate and the blood stream. These exchanges across the patient's peritoneum result in the removal of waste products, including solutes like urea and creatinine, from the blood, and regulate the levels of other substances, such as sodium and water, in the blood. The spent dialysate is then drained out of the body and discarded.

Peritoneal dialysis machines, called cyclers, are designed to assist with the procedures to infuse, allow dwelling of, and drain dialysate to, within, and from the patient's peritoneal cavity during a dialysis treatment. The treatment typically lasts for several hours, often beginning with an initial drain procedure to empty the peritoneal cavity of any remaining used or spent dialysate. The sequence then proceeds through the succession of fill, dwell, and drain phases, or cycles, that follow one after the other.

Peritoneal dialysis can be performed either using an automated pumping mechanism or using gravity to assist with filling and draining the peritoneal cavity during a PD treatment. Fully automated peritoneal dialysis (APD) cyclers use pumps to control fluid flow for the fill and drain cycles. In some circumstances, the noise produced by the operating pumps may inconvenience the patient, particularly at night. In contrast, PD gravity-based cyclers use a gravity-based fluid control system that may operate more silently than a pump-based APD cycler. In a gravity-based cycler system, dialysate is contained in a plastic bag or other container, that is to be attached via tubing to the patient's catheter, and raised to a sufficient height (e.g., above the shoulder) so that gravity may be used to continuously pull the solution down into the patient's abdomen during the fill cycle. During the drain cycle, gravity is allowed to pull solution from the patient's abdomen to a lower-positioned bag or drain. Because the therapy is gravity dependent, the peritoneal dialysis solution bag must be raised to a height sufficient to provide the potential energy necessary to infuse the peritoneal dialysis solution into the patient during the fill cycle. The greater the difference in height, the faster the inflow. If the patient is standing or lying in a tall bed, the height differential may not be enough to perform the inflow in an appropriate amount of time. Some configurations further use an even taller IV pole hook to which additional supply bags must be hoisted.

Dialysis patients with co-morbidities and lifting restrictions may have difficulty lifting solution bags above chest height, especially when the solution bag has a significant weight (e.g., as much as 6 kg for a 6 L dialysis solution bag). Additionally, beds for pediatric patients are often much higher than typical beds, which thereby require the solution bag to be raised to further heights, making gravity-based cyclers a less workable option.

Prior attempts to resolve these issues include structures with a mast on which the dialysis solution bags can be raised to the desired height after loading them at a comfortable level for the patient. Some such systems include a motor to drive a chain that raises a platform to the top of a mast, whereas others use a cable and winch that can be cranked by the user to raise and lower the control unit/head along the mast. These systems have the disadvantage of requiring a tall pillar as the structure, with the length of the pillar being the minimum length the entire device can be compressed when transporting it. In addition, because peritoneal dialysis patients may want to travel with their cyclers, conventional gravity-based cyclers may often need to be transported in smaller sections to be assembled on location before treatment can begin.

It is with respect to these and other considerations that the present disclosure may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In accordance with this disclosure, support systems and methods for adjusting a height of a gravity-based peritoneal dialysis device are provided. In some embodiments, a support system for a gravity-based peritoneal dialysis device includes a base, a support platform, and an adjustable pillar assembly connected between the base and the support platform. The adjustable pillar assembly can have a total length that is adjustable to correspondingly adjust a height of the support platform relative to the base.

In another embodiment, a method for adjusting a height of a gravity-based peritoneal dialysis device includes connecting a support platform to a first end of an adjustable pillar assembly, connecting a base to a second end of the adjustable pillar assembly, the second end substantially opposing the first end, and adjusting a total length of the adjustable pillar assembly to correspondingly adjust a height of the support platform relative to the base.

In any preceding or subsequent example, the adjustable pillar assembly can include a telescoping pole configuration comprising two or more pillar elements that are selectively movable relative to one another to change the total length of the adjustable pillar assembly. In some examples, the two or more pillar elements are coupled to a drive mechanism that is operable to selectively move the two or more pillar elements relative to one another. Examples of a drive mechanism can include a gas lifting cylinder, a hydraulic piston, a spring-assisted scissor lift mechanism, or a linear actuator.

In any preceding or subsequent example, the adjustable pillar assembly can include a first arm that extends from the support platform toward the base, a second arm that extends from the base toward the support platform, a connecting cable coupled between an end of the first arm and an end of the second arm, and a plurality of peripheral support cables coupled directly between the support platform and the base. In this configuration, a length of the connecting cable and lengths of the plurality of peripheral support cables can be adjustable to change the total length of the adjustable pillar assembly.

In any preceding or subsequent example, the connecting cable can be fixedly connected to the end of the first arm, while the connecting cable is selectively extendable and retractable relative to the end of the second arm. In some examples, the connecting cable extends from a cable supply arranged at or within the base and is routed along or through the second arm to the end of the second arm. For instance, the cable supply can be a cable spool that is rotatable to selectively extend or retract the connecting cable. In some examples, the plurality of peripheral support cables are coupled to the cable spool such that rotation of the cable spool to selectively extend or retract the connecting cable causes a corresponding retraction or extension, respectively, of the peripheral support cables.

In contrast to conventional gravity-based cyclers, which are designed as tall structures that require patients to lift solution bags up and must be painstakingly assembled from multiple pieces before use, the present systems provide a support structure that both has an adjustable height and is collapsable to a comparatively small configuration for storage and/or transportation. In this way, the present systems and methods can provide an ease of use for those who need it the most, which can lead to an increase in the adoption of gravity-based cyclers in a wider variety of markets.

Although some of the features of the present disclosure have been stated hereinabove, and which are achieved in whole or in part, other feature will become evident as the description proceeds when taken in connection with the accompanying drawings as best described hereinbelow.

### BRIEF DESCRIPTION OF THE DRAWINGS

The features and advantages of the present disclosure will be more readily understood from the following detailed description which should be read in conjunction with the accompanying drawings that are given merely by way of explanatory and non-limiting example, and in which:

FIG. 1 is a side view of a system according to one or more features of the present disclosure for supporting a gravity-based peritoneal dialysis device at any of a range of heights.

FIG. 2 is a side perspective view of an embodiment for an adjustable pillar assembly for a support system having a telescoping pole configuration according to one or more features of the present disclosure.

FIGS. 3A and 3B are side schematic views of another embodiment of an adjustable support system having a tensegrity configuration shown in different operating positions according to one or more features of the present disclosure.

It should be understood that the drawings are not necessarily to scale and that the disclosed embodiments are sometimes illustrated diagrammatically and in partial views. In certain instances, details which are not necessary for an understanding of the disclosed methods and devices, or which render other details difficult to perceive, may have been omitted. It should be further understood that this disclosure is not limited to the particular embodiments illustrated herein. In the drawings, like numbers refer to like elements throughout unless otherwise noted.

### DETAILED DESCRIPTION

The present disclosure provides systems and methods for adjusting a height of a peritoneal dialysis gravity-based cycler. Various features or the like of an adjustable gravity-based cycler support system will now be described more fully herein with reference to the accompanying drawings, in which one or more features of the adjustable gravity-based cycler support system will be shown and described. It should be appreciated that the various features may be used independently of, or in combination, with each other. It will be appreciated that the adjustable gravity-based cycler support system as disclosed herein may be embodied in many different forms and may selectively include one or more concepts, features, or functions described herein. As such, the adjustable gravity-based cycler support system should not be construed as being limited to the specific examples set forth herein. Rather, these examples are provided so that this disclosure will convey certain features of the adjustable gravity-based cycler support system to those skilled in the art.

In accordance with one or more features of the present disclosure, the present disclosure provides systems and methods for supporting a gravity-based peritoneal dialysis cycler at any of a range of heights. In some embodiments, it is desirable for the height range to be greater than about 24 inches (e.g., between about 24 inches and about 48 inches). Referring to an example embodiment shown in FIG. 1, a support system for this purpose, generally designated 100, includes a support platform 102, a base 104, and an adjustable pillar assembly 110 connected therebetween. It is contemplated that in other embodiments the system described herein may be implemented using one or more adjustable pillar assemblies like the adjustable pillar assembly 110. In some embodiments, the support platform 102 is particularly sized and configured to receive thereon a gravity-assisted cycler 10. In some embodiments, the gravity-assisted cycler 10 includes a solution bag receiver 12 that is configured to receive one or more units of prepared peritoneal dialysis solution. The adjustable pillar assembly 110 can be connected to a drive mechanism 105, as an external component or integrated within the adjustable pillar assembly 110, which can be selectively operable to change a total length of the adjustable pillar assembly 110. A control device 106 may be coupled to or integrated with the drive mechanism 105 to enable a patient or other user to control or manipulate the drive mechanism 105. For example, in an embodiment, the control device 106 may include manual interfaces or buttons to engage the drive mechanism. Additionally and/or alternatively, in other embodiments, the control device 106 may include a digital controller that may be engaged using a wirelessly coupled device, such as a smartphone or other electronic device.

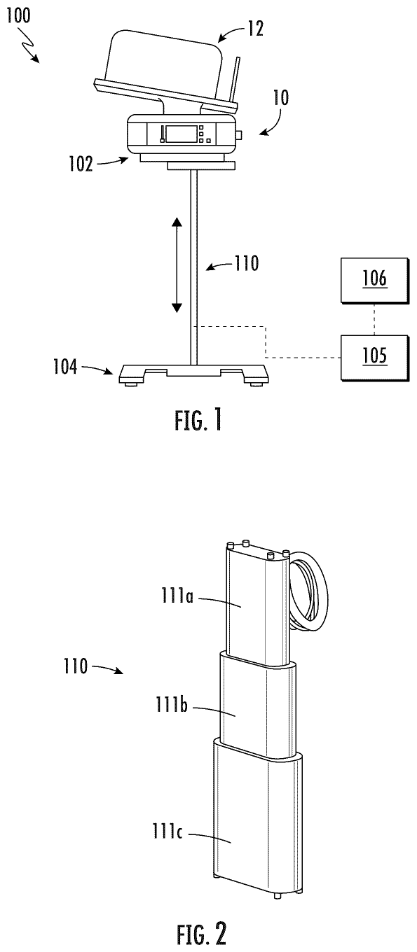

In one configuration shown in FIG. 2, the adjustable pillar assembly 110 is provided in a telescoping pole configuration that can be easily raised and lowered by a peritoneal dialysis patient at any time during use. In the illustrated embodiment, the adjustable pillar assembly includes three pillar elements: a first pillar element 111a, a second pillar element 111b, and a third pillar element 111c. Those having ordinary skill in the art will recognize, however, that more or fewer pillar elements can be included in the adjustable pillar assembly 110. In any configuration, the pillar elements are hollow tube elements having different sizes such that sequentially smaller ones of the pillar elements are configured to nest within one another and are selectively movable relative to one another, such as in a telescoping manner, to change a total length of the adjustable pillar assembly 110.

The drive mechanism 105 can be provided in any of a variety of forms to drive the selective adjustment of the total height. In some embodiments, the drive mechanism 105 can include a gas lifting cylinder coupled to the adjustable pillar assembly 110 to raise the gravity-assisted cycler 10 and solution bags above it. In some other embodiments, the drive mechanism 105 can include a hydraulic piston that can be pumped up to raise the gravity-assisted cycler 10, and a lever can be operated to lower the unit again. In some other embodiments, the drive mechanism 105 can include a spring configured to provide tension to scissor-lift the gravity-assisted cycler 10 in a manner similar to a spring-assisted adjustable height workstation. In any of these types of configurations, the selected height can be locked in place after a patient raises the platform and can be unlockable only while gripping the lift mechanism.

In other configurations, the drive mechanism 105 can include a linear actuator mounted within or otherwise coupled to the adjustable pillar assembly 110. In some embodiments, a single pillar is extended using a screw-driven shaft, which can be powered, for example, by a motor. In this way, such a configuration can operate in a manner similar to a motorized standing desk. In further configurations, the drive mechanism 105 can include a rack-and-pinion type of drive system in which a linear, flexible toothed structural element is pushed/pulled from a motorized rotational gear.

Regardless of the particular mechanism that is used to adjust the total length of the adjustable pillar assembly 110, the adjustable support system 100 provides convenience and ease of use in operation while also providing the ability to collapse the support system 100 into a smaller space for storage or transport.

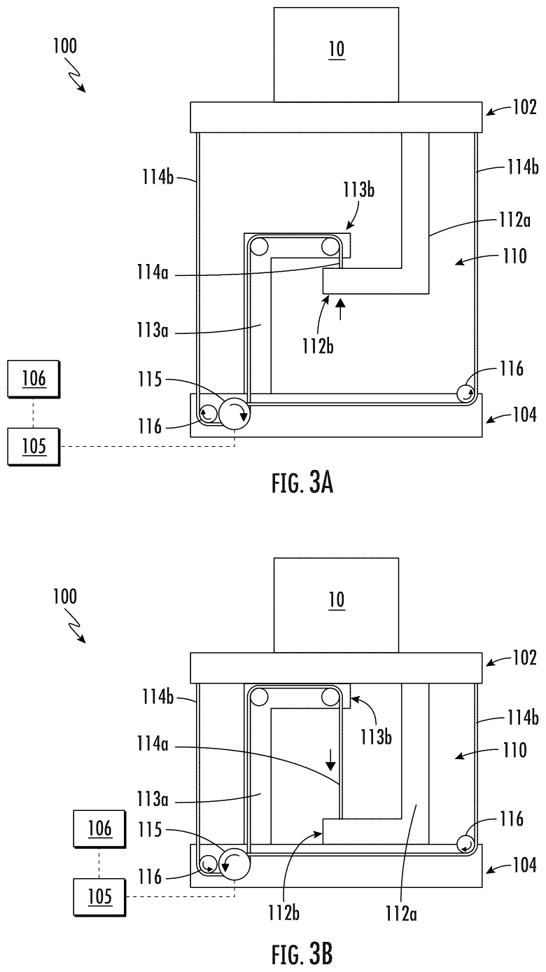

In accordance with one or more further features of the present disclosure, the adjustable pillar assembly 110 can have a static tensegrity configuration that utilizes vertical tension to suspend the support platform 102 above the base 104. Referring to FIGS. 3A and 3B, the support system 100 having a tensegrity configuration can include a first arm 112a that extends downward from the support platform 102 toward the base 104, and the base 104 includes a second arm 113a that extends upward toward the support platform 102. An end 112b of one of the first arm 112a can be coupled to an end 113b of the second arm 113a by a connecting cable 114a. It is contemplated herein that in other embodiments the system described herein may be implemented using multiple first and/or second arms like the first arm 112a and the second arm 113a.

In the configuration illustrated in FIGS. 3A and 3B, the position at which the first arm 112a is connected to the support platform 102 is offset with respect to the position at which the second arm 113a is connected to the base 104, but the end 112b of the first arm 112a is angled toward the second arm 113a, and/or the end 113b of the second arm 113a is angled toward the first arm 112a. In this arrangement, the end 113b of the second arm 113a can be positioned closer to the support platform 102 than the end 112b of the first arm 112a (and the end 112b of the first arm 112a can correspondingly be positioned closer to the base 104 than the end 113b of the second arm 113a), with the connecting cable 114a extending substantially vertically between the ends 112b and 113b. In this arrangement, the length of the connecting cable 114a is inversely related to the height of the support platform 102 relative to the base 104.

In some embodiments, the support system 100 further includes a plurality of peripheral support cables 114b that directly connect the support platform 102 to the base 104. In the illustrated embodiment, the support platform 102 and the base 104 each have a substantially rectangular shape, and four peripheral support cables 114b are connected between corresponding corners of the support platform 102 and the base 104. Those having ordinary skill in the art will recognize, however, that more or fewer peripheral support cables 114b can be provided in different arrangements to produce a stable support configuration.

Regardless of the particular configuration, the components of this tensegrity configuration of the adjustable pillar assembly are arranged such that the first arm 112a is substantially entirely in compression between the support platform 102 and the connecting cable 114a, the second arm 113a is substantially entirely in compression between the connecting cable 114a and the base 104, the cable 113a is substantially entirely in tension between the end 112b of the first arm 112a and the end 113b of the second arm 113a, and the plurality of peripheral support cables 114b are substantially entirely in tension between the support platform 102 to the base 104. Those having ordinary skill in the art will recognize that the support platform 102 can thereby be stably held at a desired height about the base 104 depending on the lengths of the first arm 112a, the second arm 113a, the connecting cable 114a, and the peripheral support cables 114b.

In some embodiments, the length of the connecting cable 114a and the lengths of the peripheral support cables 114b are adjustable to correspondingly adjust the height of the support platform 102 relative to the base 104. Specifically, for example, by shortening the connecting cable 114a and correspondingly lengthening the peripheral support cables 114b, the end 112b of the first arm 112a can be pulled closer to the end 113b of the second arm 113a such that the height of the support platform 102 can be increased relative to the base 104. In such a configuration, the support platform 102 can be movable to a minimum height relative to the base 104 at which the connecting cable 114a is effectively fully extended such that the first arm 112a extends effectively the entire distance from the support platform 102 to the base 104 (and/or the second arm 113a extends effectively the entire distance from the base 104 to the support platform 102), such as is shown in FIG. 3B. The support platform 102 can also be movable to a maximum height relative to the base 104 at which the connecting cable 114a is effectively fully retracted such that a distance between the end 112b of the first arm 112a and the end 113b of the second arm 113a is minimized, such as is shown in FIG. 3A.

Referring again to the embodiment illustrated in FIGS. 3A and 3B, the connecting cable 114a can be fixedly connected to the end 112b of the first arm 112a, but the connecting cable 114a can be selectively extendable and retractable relative to the end 113b of the second arm 113a. In some embodiments, the connecting cable 114a extends from a cable supply 115 arranged at or within the base 104 and is routed along or through the second arm 113a (e.g., by one or more pulleys), to the end 113b of the second arm 113a, and toward the end 112b of the first arm 112a. Alternatively, the cable supply 115 can be arranged in or on the support platform 102. In some embodiments, the connecting cable 114a is enclosed within the second arm 113a for preventing pinch points, for providing additional stability, and/or for aesthetic reasons.

In some embodiments, the cable supply 115 is a cable spool or ratcheting winch that is rotatable to selectively extend (i.e., unspool) or retract (i.e., re-spool) the connecting cable 114a. In some embodiments, the drive mechanism 105 can include a hand crank that is connected to the cable supply 115 and is manually operable to adjust the length of the connecting cable 114a. Alternatively, the drive mechanism 105 can include a motor or other controllable mechanical actuator that is connected to the cable supply 115, allowing the patient to raise and lower the platform, such as by engaging the control device 106, for example with the touch of a button or operation of an application running on a personal electronic device.

In some embodiments, the peripheral support cables 114b can also be coupled to the cable supply 115 such that extension or retraction of the connecting cable 114a is performed in coordination with a corresponding retraction or extension, respectively, of the peripheral support cables 114b. In some embodiments, a series of peripheral pulleys 116 can route the peripheral support cables 114b to the cable supply 115.

Alternatively, in some embodiments, the peripheral support cables 114b each extend from a secondary cable supply that is operable in parallel with the cable supply 115 to coordinate the adjustment of the lengths of the respective cables. In some embodiments, the tension of each of the peripheral support cables 114b can be independently adjusted with respect to one another and/or with respect to the tension of the connecting cable 114a. This independent operability can allow the support system 100 to adjust the balance of forces in the structure, such as to provide more tension on selected ones of the peripheral support cables 114b to compensate for imbalances in the loading on the support platform 102 (e.g., if the solution bags are loaded off-center).

In some embodiments, one or more sensors, such as a tilt sensor or accelerometer, can be provided in communication with the support platform 102 to detect instability of the structure. In situations in which the support platform 102 is deemed to be unstable, the adjustable pillar assembly 110 can be configured to automatically adjust the tension of the connecting cable 114a and/or the peripheral support cables 114b or to trigger an automatic lowering of the adjustable pillar assembly 110. In some examples, such automatic adjustment can be triggered by one or more proximity sensor arranged to detect patient limbs or other obstructions that are present when the support platform 102 is moved.

In any configuration, the support system 100 can support a substantial amount of weight based on the tensile strength of the connecting cable 114a and the peripheral support cables 114b and the compressive strength of the first arm 112a and the second arm 113a. In addition, because a robust frame is already required for a conventional gravity-based cycler support, this implementation adds very little weight compared to traditional structures.

In any of the disclosed configurations, the peritoneal dialysis solution bags can be loaded at a low height, but they can then be raised to a height selected to provide a desired treatment height differential. This adjustability can give patients the freedom of operation to take advantage of a gravity-based cycler's quiet operation.

In addition, in some embodiments, the height can be automatically adjusted based on the stage of operation to optimize the performance of the peritoneal dialysis gravity-based cycler. In some embodiments, for example, the support platform 102 can be raised during inflow of the peritoneal dialysis solution in the fill cycle and lowered during outflow in the drain cycle. In some embodiments, the gravity-assisted cycler 10 can be configured to receive feedback via one or more sensors indicating the fluid pressure or resistance during each stage, and the height of the support platform 102 can be automatically adjusted in response to this feedback. In some embodiments, for example, the height of the support platform 102 can be controlled in a manner to maintain a desired flow rate of fluid passing from the dialysis machine to the patient or vice versa.

In some embodiments, for example, the support platform 102 can be automatically raised or lowered based on the particular type of user interaction with the device. In some examples, the support platform 102 can be configured to drop during setup to a lowest height to facilitate easier loading of bags. Alternately, the support platform 102 can be configured to automatically raise to a treatment inflow height after setup. Further, in some embodiments, if the connection to the elevated supply bag is singled out in an alarm situation that requires troubleshooting, the support platform 102 can be configured to automatically descend to a lower height so the patient would have easier access to inspect the connection.

The present disclosure is presented principally in connection with a peritoneal dialysis gravity-based cycler system. However, it should be understood that the features and functions of the system described herein may be used and/or implemented in connection with other medical devices and applications in which an adjustable support system to enable height adjustment for gravity-based fluid transfer would be beneficial.

The present disclosure can be embodied in other forms without departure from the spirit and essential characteristics thereof. The embodiments described therefore are to be considered in all respects as illustrative and not restrictive. Although the present disclosure has been described in terms of certain preferred embodiments, other embodiments that are apparent to those of ordinary skill in the art are also within the scope of the present disclosure.

As used herein, an element or operation recited in the singular and proceeded with the word “a” or “an” should be understood as not excluding plural elements or operations, unless such exclusion is explicitly recited. Furthermore, references to “one embodiment” or “one example” of the present disclosure are not intended to be interpreted as excluding the existence of additional embodiments or examples that also incorporate the recited features.

The present disclosure is not to be limited in scope by the specific embodiments or examples described herein. Indeed, other various embodiments or examples and modifications to the present disclosure, in addition to those described herein, will be apparent to those of ordinary skill in the art from the foregoing description and accompanying drawings. Thus, such other embodiments or examples and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A support system for a gravity-based peritoneal dialysis device, the system comprising:
a base;
a support platform; and
an adjustable pillar assembly connected between the base and the support platform, the one or more adjustable pillar assembly having a total length that is adjustable to correspondingly adjust a height of the support platform relative to the base;
wherein the adjustable pillar assembly comprises a telescoping pole configuration comprising two or more pillar elements that are selectively movable relative to one another to change the total length of the adjustable pillar assembly.

2. The support system of claim 1, wherein the two or more pillar elements are coupled to a drive mechanism that is operable to selectively move the two or more pillar elements relative to one another.

3. The support system of claim 2, wherein the drive mechanism is selected from the group consisting of: a gas lifting cylinder, a hydraulic piston, a spring-assisted scissor lift mechanism, and a linear actuator.

4. A support system for a gravity-based peritoneal dialysis device, the system comprising:
a base;
a support platform; and
an adjustable pillar assembly connected between the base and the support platform, the adjustable pillar assembly having a total length that is adjustable to correspondingly adjust a height of the support platform relative to the base, wherein the adjustable pillar assembly comprises:
a first arm that extends from the support platform toward the base;
a second arm that extends from the base toward the support platform;
a connecting cable coupled between an end of first arm and an end of the second arm; and
a plurality of peripheral support cables coupled directly between the support platform and the base;
wherein a length of the connecting cable and lengths of the plurality of peripheral support cables are adjustable to change the total length of the adjustable pillar assembly.

5. The support system of claim 4, wherein the connecting cable is fixedly connected to the end of the first arm; and
wherein the connecting cable is selectively extendable and retractable relative to the end of the second arm.

6. The support system of claim 5, wherein the connecting cable extends from a cable supply arranged at or within the base and is routed along or through the second arm to the end of the second arm.

7. The support system of claim 6, wherein the cable supply comprises a cable spool that is rotatable to selectively extend or retract the connecting cable.

8. The support system of claim 7, wherein the plurality of peripheral support cables are coupled to the cable spool such that rotation of the cable spool to selectively extend or retract the connecting cable causes a corresponding retraction or extension, respectively, of the peripheral support cables.

9. The method for adjusting a height of a gravity-based peritoneal dialysis device, the method comprising:
connecting a support platform to a first end of an adjustable pillar assembly;
connecting a base to a second end of the adjustable pillar assembly, the second end substantially opposing the first end; and
adjusting a total length of the adjustable pillar assembly to correspondingly adjust a height of the support platform relative to the base.

10. The method of claim 9, wherein the adjustable pillar assembly comprises a telescoping pole configuration comprising two or more pillar elements; and
wherein adjusting a total length of the adjustable pillar assembly comprises selectively moving the two or more pillar elements relative to one another to change the total length of the adjustable pillar assembly.

11. The method of claim 10, wherein selectively moving the two or more pillar elements comprises operating a drive mechanism to selectively move the two or more pillar elements relative to one another, wherein the drive mechanism is selected from the group consisting of a gas lifting cylinder, a hydraulic piston, a spring-assisted scissor lift mechanism, and a linear actuator.

12. The method of claim 9, wherein connecting the support platform to the first end of the adjustable pillar assembly comprises connecting a first arm to the support platform, the first arm extending from the support platform toward the base;
wherein connecting the base to the second end of the adjustable pillar assembly comprises connecting a second arm to the base, the second arm extending from the base toward the support platform;
wherein the one or more adjustable pillar assembly comprises:
a connecting cable coupled between an end of the first arm and an end of the second arm; and
a plurality of peripheral support cables coupled directly between the support platform and the base;
wherein adjusting a total length of the one or more adjustable pillar assembly comprises adjusting a length of the connecting cable and lengths of the plurality of peripheral support cables to change the total length of the one or more adjustable pillar assembly.

13. The method of claim 12, wherein the connecting cable is fixedly connected to the end of the first arm; and
wherein the connecting cable is selectively extendable and retractable relative to the end of the second arm.

14. The method of claim 13, wherein adjusting a length of the connecting cable comprises selectively extending or retracting the connecting cable with respect to a cable supply arranged at or within the base.

15. The method of claim 14, wherein the cable supply comprises a cable spool; and
wherein selectively extending or retracting the connecting cable comprises rotating the cable spool.

16. The method of claim 15, wherein the plurality of peripheral support cables are coupled to the cable spool; and
wherein rotating the cable spool to selectively extend or retract the connecting cable causes a corresponding retraction or extension, respectively, of the peripheral support cables.
